(19) United States
(12) Patent Application Publication
(10) Pub. No.: US 2005/0203413 A1
(43) Pub. Date: Sep. 15, 2005

(54) Transcavital needle insertion device

(76) Inventors: Gabor Fichtinger, Bethesda, MD (US); Allison M. Okamura, Towson, MD (US); Chad M. Schneider, Pikesville, MD (US)

Correspondence Address: MCKENNA LONG & ALDRIDGE LLP, 1900 K STREET, NW, WASHINGTON, DC, 20006, US

(21) Appl. No.: 10/912,545

(22) Filed: Aug. 6, 2004

Related U.S. Application Data
(60) Provisional application No. 60/493,406, filed on Aug. 7, 2003.

(51) Int. Cl. A61B 8/14

(52) U.S. Cl. 600/461

(57) ABSTRACT

Disclosed is a transcavital needle insertion device that incorporates a transrectal ultrasound (TRUS) probe; a support sheath incorporated with, but mechanically decoupled from the TRUS probe to substantially stabilize the target tissue being imaged; and a needle guide sheath that moves relative to the TRUS probe. The device substantially enables a practitioner to more accurately and precisely insert a therapeutic needle into a target tissue, such as a prostate, in a decoupled three degree of freedom coordinate space that is registered to the imagery generated from the TRUS probe. The support sheath may enable the practitioner to move the TRUS probe, and independently position and insert the needle, without problems brought about by variable deformation of the target tissue, which would otherwise result from motion of the TRUS probe and the needle.

This application claims the benefit of U.S. Provisional Patent Application No. 60/493,406, filed on Aug. 7, 2003, which is hereby incorporated by reference for all purposes as if fully set forth herein.

Research and development efforts associated with the subject matter of this patent application was supported by the National Science Foundation under Grant No. EEC-9731478.

### BACKGROUND OF THE INVENTION

1. Field of the Invention

The present invention relates to controllable transcavital needles and catheters and their use in endo-cavital surgery. More particularly, the present invention relates to transcavital needle guides incorporated with ultrasonic probes to provide minimally invasive transrectal prostate treatment with more accurate needle targeting.

2. Discussion of the Related Art

Prostate cancer is the second leading cause of cancer death among American men, claiming 30,000 lives per year in the United States. Close to one million prostate biopsies are performed in the U.S. annually, and the estimated number of new prostate cancers detected in 2002 was 189,000. In addition to cancer, about 50% of men over 50 years old in the United States experience symptoms from Benign Prostate Hyperplasia, the enlargement of the prostate that can result in acute urinary retention and require surgery if left untreated.

In contemporary practice, prostate biopsy and most local therapies are executed via needles inserted into the prostate through the perineum or through the rectal wall. Both access routes have been documented to be safe and well tolerated. High Intensity Interstitial Ultrasound (HIIU) tissue ablator needles are used in a similar manner for prostate therapy. There are several factors in deciding the optimal access route for any given prostatic needle intervention: the number of insertions, needle placement error, need for anesthesia, and risk of infection. Generally, for interventions involving only a limited number of needle insertions, like biopsy, transrectal access is preferable. Transrectal ultrasound (TRUS) has been the dominant imaging modality in the guidance of prostate biopsy and therapeutic interventions. In current practice, however, the probe is manipulated freehand inside the rectum, thereby causing variable deformation to the prostate and rendering transrectal needle placement imprecise and unpredictable.

In related art practice, variable deformation of the prostate may occur due to variable normal forces imparted by the ultrasound probe in a manner similar to that caused by needle insertion. This deformation may interfere with the registration of the ultrasound imagery to the target tissue into which the practitioner inserts the needle. Such variable deformation problems related to ultrasound may occur during any transrectal ultrasound (TRUS) procedure.

Accordingly, there is a need to more accurately and precisely guide one or more needles into a prostate by entering through the rectal cavity wall. Precise needle insertion is made difficult by factors including needle deflection through tissue interaction, and variable deformation of the prostate during needle insertion and ultrasound imaging. As such, there is a need for way to stabilize the target tissue during both ultrasonic imaging and needle insertion. Such stability would help to assure ultrasonic image registration, which would improve the accuracy and precision of inserting therapeutic needles into a target prostate tissue. Further, there is a need to more accurately and precisely guide one or more therapeutic needles into the prostate without interfering with the surrounding anatomy.

### SUMMARY OF THE INVENTION

Accordingly, the present invention is directed to a transcavital needle placement device that substantially obviates the deficiencies and disadvantages associated with the related art as set forth above. More specifically, the present invention is directed to a controllable and movable needle guide that, in conjunction with a medical imaging system, enables more accurate placement of a transcavital needle without the position uncertainty, possible tissue damage, and imaging anomalies brought on by problems associated with related art.

It should be noted that the following detailed description portrays various exemplary embodiments of the present invention as being particularly useful in the field of prostate cancer treatment. However, it will be very clear to one skilled in the art that the present invention will be equally applicable to other natural and artificial body cavities in addition to the rectum, to other organs in addition to the prostrate, and to other image guidance modalities in addition to ultrasound. Further, it will be readily apparent that the present invention may be used to guide the insertion of catheters, laparoscopic tools with optical fiber video features, other types of probes, or a variety of other devices. Also, in place of the ultrasound probe described below, a laparoscopic ultrasound probe, or some other probe, could be used.

As such, one advantage of the present invention is to provide for more accurate guidance of one or more therapeutic needles during prostate treatment.

Another advantage of the present invention is to provide more accurate image guided placement of needles or probes during transcavital surgery.

Another advantage of the present invention is to improve the placement of therapeutic needles while avoiding certain surrounding anatomy.

Yet another advantage of the present invention is to provide for more effective use of a therapeutic needle by striking an advantageous balance between the preferred angle of entry into a cavity wall and the flexibility of the needle material.

To achieve these and other advantages and in accordance with the purpose of the present invention, as embodied and described, a transcavital needle insertion device comprises: a support sheath; an ultrasound probe; and a guide sheath having at least one needle guide.

In another aspect of the present invention, a method for inserting a needle into a cavity wall using a transcavital needle insertion device having a support sheath, an ultrasound probe, and a guide sheath having a needle guide, the method comprises the steps of: inserting the transcavital needle device into a cavity; obtaining an ultrasound image; determining a target location inside the cavity wall; computing a guide sheath position corresponding to the target location; computing a needle depth corresponding to the target location and the guide sheath position; positioning the guide sheath according to the guide sheath position; and inserting the needle according to the needle depth.

In another aspect of the present invention, a computer readable medium encoded with a program for controlling a transcavital needle insertion device, the device having a guide sheath, a guide sheath positioner, and a needle depth positioner, the program comprises the steps of: acquiring a desired needle tip position; converting the desired needle tip position to a desired translational position for the guide sheath, a desired rotational position for the guide sheath, and a desired needle depth; sending a command to the guide sheath positioner corresponding to the desired translation position; sending a command to the guide sheath positioner corresponding to the desired rotational position; and sending a command to the needle depth positioner corresponding to the desired needle depth.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, together with the detailed description below, set forth the various aspects and embodiments of the present invention, wherein:

FIG. 1 is a diagram of an exemplary system according to the present invention;

FIG. 2 is a diagram of an exemplary device of the present invention;

FIG. 3 shows an exemplary guide sheath;

FIG. 4 shows an exemplary support sheath according to the present invention;

FIG. 5 illustrates an exemplary process for using the present invention in a surgical procedure;

FIG. 6A is a geometric diagram of the guide sheath and the needle for calculating an inverse kinematic solution, with the longitudinal axis going into the page;

FIG. 6B is a diagram of the guide sheath and the needle, with the longitudinal axis parallel to the page;

FIG. 7 is an axial view of the reachable workspace inside the prostate, along with exemplary needle positions according to the present invention; and

FIG. 8 shows another exemplary process for using the present invention in a surgical procedure.

### DETAILED DESCRIPTION OF THE ILLUSTRATED EMBODIMENTS

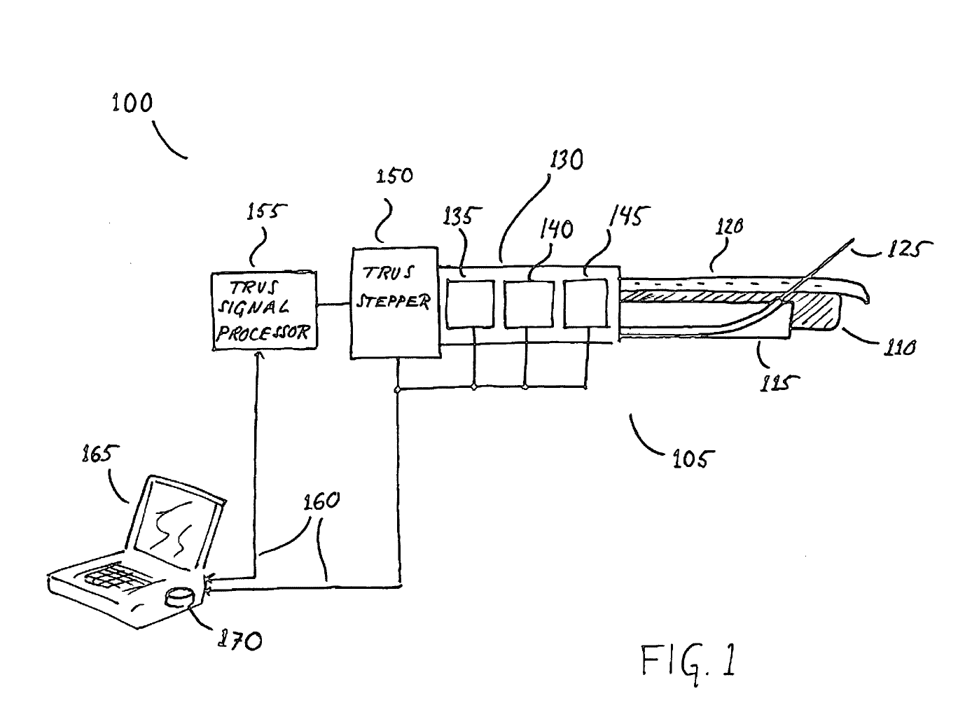

FIG. 1 shows a system 100 for inserting one or more needles 125 into a prostate for the purposes of cancer treatment. The system includes a transcavital needle placement device 105, which comprises a transrectal ultrasound (TRUS) probe 110; a TRUS stepper 150; a needle guide sheath 115; and a set of positioners 130, which includes a guide sheath rotational positioner 145; a guide sheath translational positioner 140; and a needle depth positioner 135. In addition to the device 105, the system 110 includes a TRUS signal processor 155, and a computer 165, which stores and executes software 170 according to the present invention.

For the purposes herein, “positioner” includes any device or devices for establishing and measuring the position of the guide sheath 115 or the needle 125. For example, a positioner may include a motor, a gear mechanism, and an encoder. The motor may be an electric motor. Alternatively, the positioner may include a handle in place of the motor, and may be operated manually. The encoder may preferably be an optical encoder, although other types of position and angle measurement devices may be used. In an exemplary embodiment, the guide sheath translational positioner 140 may use a geared drive with an encoded DC motor, and may include a DC, 6 Watt, A-max 22 graphite brush-type motor with a maximum torque of 7.19 mNm, combined with a planetary gearhead GP 22 A, 19:1, and a 2-channel 100 count/turn digital encoder, like that commercially available from Maxon Precision Motors (Burlingame, Calif.). A suitable motor may be selected by estimating the force of friction and interaction forces with the tissue on the guide sheath 115, and combining these forces with the ratios all of the rotational linkages to determine a rough specification for the motor.

Generally, the resolution of the encoders may be selected based on the precision requirements pertaining to the target location in the prostate. For example, a preferred resolution for measuring the guide sheath 115 angle is approximately 0.1 degrees, and a preferred resolution for measuring the translation of the guide sheath 115 and the depth of the needle 125 is approximately 0.1 mm. The positioner may include an embedded processor or microcontroller, or may be controlled remotely by instructions executed by the software 170. In a preferred embodiment, the software 170 retrieves position and/or angle data from the positioners 130. Each of the positioners 130 may have features independent of the others. For instance, in a preferred embodiment, the rotational positioner 145 and the translational positioner 140 of the guide sheath 115 may include motors that are operated by commands from the software 170, while the needle depth positioner 135 may be operated manually. The commands to operate each of the positioners 130 may include commands corresponding to desired angular position; desired translational position; desired angular rate; desired translational velocity; desired torque; desired force; or any combination of these.

The system 100 provides for positioning and inserting a needle 125 according to a decoupled three-degree-of-freedom (3-DOF) kinematic coordinate system whereby an arbitrary target point may be defined relative to the prostate. Assuming that the needle 125 enters the tissue at some oblique angle across the cavity wall, a target point (within the prostate, for example) may be defined by (1) translation D_{d} of the needle guide sheath 115 along an axis collinear with the centerline of the TRUS probe (hereinafter the “longitudinal axis”); (2) rotation ψ of the guide sheath 115 about the longitudinal axis; and (3) depth of insertion N_{d} of the needle 125. The present invention substantially provides for more precise and accurate positioning of the needle, using this coordinate space.

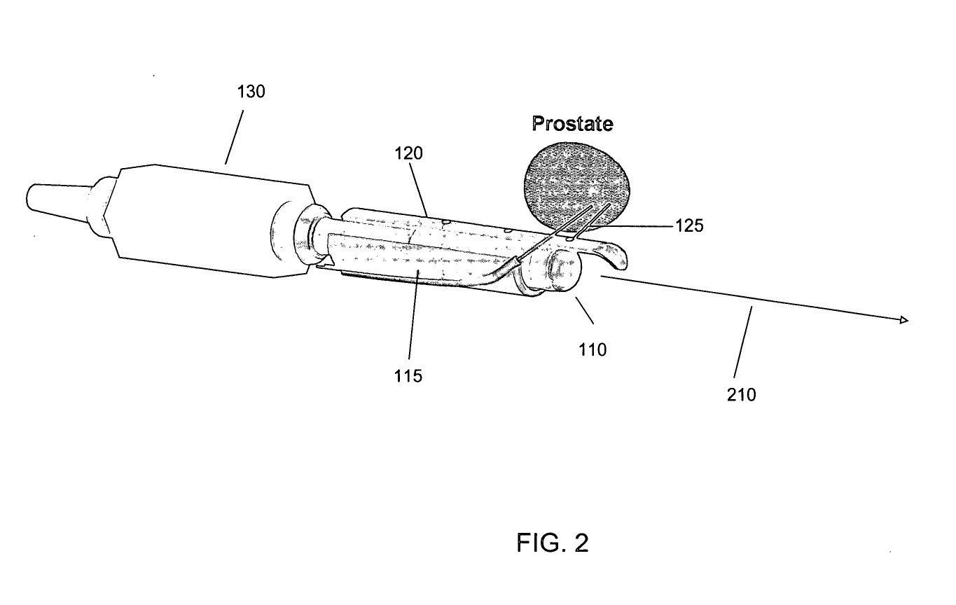

FIG. 2 shows a preferred embodiment of the device 105. Within the device 105, the TRUS probe 110 may freely move relative to the support sheath 120, and may translate along the longitudinal axis 210 according to a force applied by the TRUS stepper 150. The guide sheath 115, which is substantially collinear with the TRUS probe 110, may translate along the longitudinal axis 210 according to a force applied by the guide sheath translational positioner 140 and rotate around the longitudinal axis 210 according to a torque applied by the rotational positioner 135. The guide sheath 115 moves substantially independently of the TRUS probe 110. The support sheath 120 may preferably be rigidly affixed to the TRUS stepper base 150, or some other substantially fixed reference point in the system 100, to substantially stabilize the target tissue surrounding the prostate while the TRUS probe 110 and the guide sheath 115 are being moved.

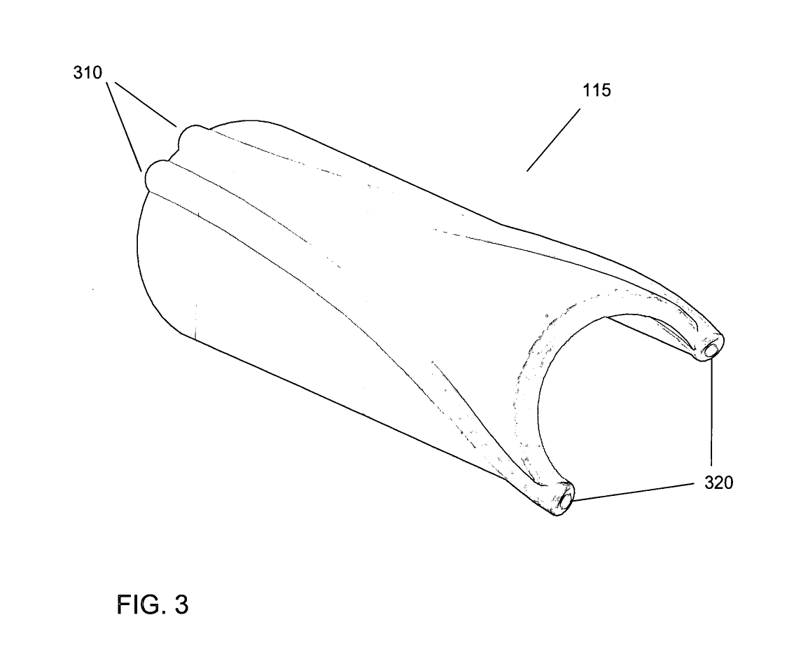

FIG. 3 shows an exemplary guide sheath 115 according to the present invention. The guide sheath 115 has at least one needle guide 310 disposed on the outer surface of the sheath. The needle guide 310 has an exit aperture 320, through which the needle 125 passes as it approaches the cavity wall. The needle guide 310 is preferably designed such that its radius of curvature is kept under given limits dictated by the elastic properties of the needle 125 to be inserted. Certain therapies and diagnoses may require specialized needles that have a given limited bending capability. Accordingly, the needle guide 310 may preferably have a parametric curve shape that is designed to conform to the bending capability of the needle 125. Generally, if the curvature of the needle 125 stays within its range of elasticity, the needle 125 will maintain a substantially straight and predictable trajectory as it exits the needle guide 310 through the exit aperture 320. In a preferred embodiment, the curvature of the needle guide 310 is designed such that the needle passes through the exit aperture 320 at an angle (hereinafter “exit angle”), which may be about 50° relative to the longitudinal axis 210. This exit angle represents a balance between minimizing the bending of the needle while minimizing interaction between the needle and particularly sensitive tissue, such as nerve bundles, which may be more problematic at shallower exit angles.

The exemplary guide sheath 115 has a left and a right needle guide 310, which enables two needles 125 to be directed toward the prostate from either side of the support sheath 120. Although the needle guides 310 are disposed on the outer surface of the exemplary guide sheath 115, the needle guides 310 may be integrated into the body of the guide sheath 115, or disposed on the interior surface of the guide sheath 115. Further, although the exemplary guide sheath 115 has two needle guides 310, it may have one needle guide 310, or a plurality of guides. If the guide sheath 115 has multiple needle guides 310, the needle guides 310 may have different curvatures and inner diameters, enabling the practitioner to use various needles or other devices in accordance with the present invention.

The exit aperture 320 may include a rounded shape, and may be substantially plugged with a cover. The cover, which may be plastic, along with the rounded shape of the exit aperture 320, may prevent the exit aperture 320 from cutting the cavity wall when the guide sheath 115 is moving within the cavity. The exit aperture 320 may also have rounded shape to prevent it from cutting the cavity wall.

In a preferred embodiment, the guide sheath 115, comprises a biologically compatible material such as, for example, PTFE (Polytetraflouroethylene, or Teflon) or Nylon 66. The guide sheath 115 may have a half cylinder shape, the open side of which may be open on the anterior side of the rectum to avoid degradation of the ultrasound signal from the prostate. In a preferred embodiment, the guide sheath 115 has a half cylinder shape such that the sheath encompasses about 210° of the 360° of a full cylinder. The guide sheath 115 may have an inner diameter of about 24.2 mm and an outer diameter of about 28.0 mm, with each needle guide 310 adding about 1.2 mm to the outer radius. The needle 125 may include nitinol, an alloy of nickel and titanium, which may be chosen for its elasticity.

The needle guides 310 may be spaced approximately 180° apart at the end of the guide sheath 115 opposite to the end having the exit apertures 320. The needle guides 310 may comprise stainless steel, brass, or another material sufficiently strong to withstand the bending forces of the needle. The needle guides 310 may be affixed to the guide sheath 115 using an epoxy like a Master Bond EP21ND 2-component epoxy. Preferably, the epoxy should be food grade, and have a USP Class VI certification. However, it may be preferable to have the needle guides 310 formed of the same material as the guide sheath 115, if the material has sufficient strength to bend the needle without distorting or becoming damaged.

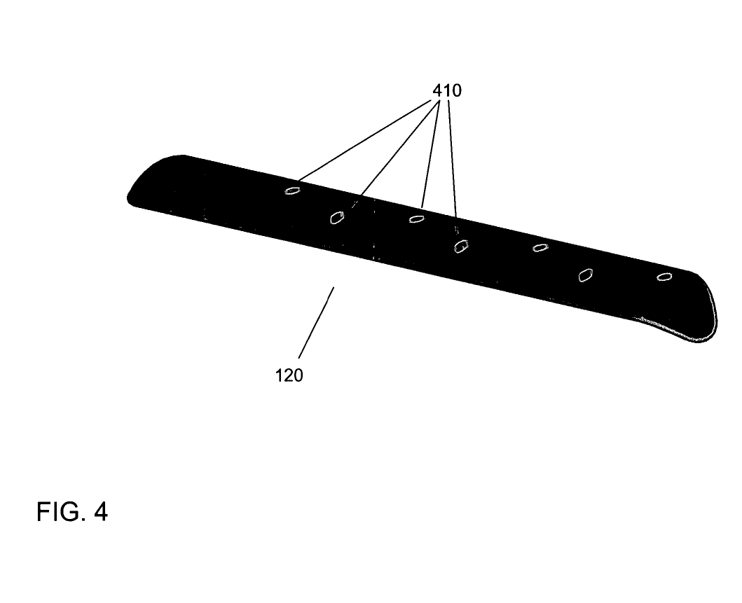

FIG. 4 shows an exemplary support sheath 120 according to the present invention. The cantilevered support sheath 120 may be rigidly affixed to the TRUS stepper 150, or to some other stationary component of the system 100, to enable the support sheath 120 may remain substantially fixed relative to the cavity wall when the practitioner adjusts the orientation of the TRUS probe 10, the guide sheath 115, and/or the needle 125 during a medical procedure. Accordingly, the support sheath 120 substantially mechanically decouples the prostate from the TRUS probe 110 and the guide sheath 115, thereby mitigating variable deformations of the prostate as the TRUS probe 110 and the guide sheath 115 are being positioned. Alternatively, the support sheath 120 may further be mechanically decoupled from the TRUS stepper 150 whereby the support sheath 120 may have its own positioner. The support sheath may comprise a biologically compatible material such as, for example, PTFE or Nylon 66.

The presence of the support sheath 120 between the TRUS probe 110 and the cavity wall may result in interference in the form of a reduction of acoustic signal coupling between the prostate and the TRUS probe 110. To mitigate this, the support sheath 120 may include a plurality of holes 410. The holes 410 may reduce the interference caused by the support sheath 120 by facilitating the flow of coupling gel during movement of the TRUS probe 110.

The TRUS stepper 150 controls the position of the TRUS probe 110. The TRUS stepper 150 may be a commercially available component, such as the Interplant® ultrasound stepper manufactured by CMS Burdette Medical Systems, IGD (St. Louis, Mo.), although other like components may be used. In a preferred embodiment, the TRUS stepper 150 provides 7-degree-of-freedom positioning control of the TRUS probe 110, while the support sheath 120 remains substantially fixed relative to the cavity wall. The TRUS stepper 150 may be controlled by the computer 165, or may have a separate user interface (not shown) for its control. Alternatively, the TRUS stepper 150 may be controlled manually. Further, the TRUS stepper may include position and angle encoders (not shown), which provide position and angle data to the computer 165, either directly from the TRUS stepper 150, or through the TRUS signal processor 155.

The TRUS probe 110 and TRUS signal processor 155 may be components of a commercially available ultrasound system. In a preferred embodiment, the TRUS signal processor 155 includes a data interface through which processed ultrasound data may be transmitted to the computer 165.

The computer 165 may be a standalone computer, or may include a plurality of computers that are networked together. In the latter case, one or more of the computers may communicate over the internet, and may include databases hosted on remote servers. Further, one or more of the computers that make up computer 165 may be embedded processors. It will be readily apparent to one skilled in the art that many architectures are possible for the computer 165 and within the scope of the present invention.

The software 170, which is stored in and executed by the computer 165, includes computer instructions and configuration data values for controlling the components of the system 100; acquiring data from one or more components of the system 100; computing the position of the guide sheath 115 and the needle 125 relative to the prostate; registering the needle to processed ultrasound data from the TRUS signal processor 155 and displaying the corresponding images; interacting with an operator or practitioner; and storing image data values. The software 170 may be distributed among many computers, or may be stored on one computer and launched to operate on multiple computers. The software 170 may include components that interact with remote databases and remote operators over the internet. It will be readily apparent to one skilled in the art that many architectures for the software 170 are possible and within the scope of the present invention.

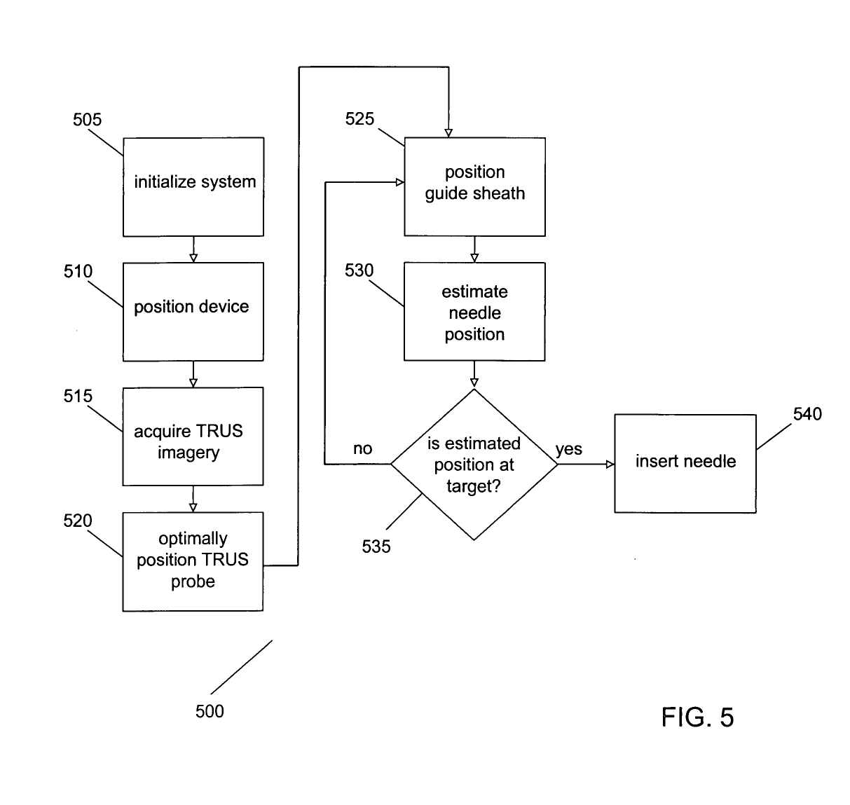

FIG. 5 illustrates an exemplary process 500 that may be implemented at least in part by the computer instructions within the software 170. The system 100 is initialized in step 505. Initialization may include, for example, initializing the positioners 130 and the TRUS stepper 150 to move to a “home” state or position; prompting the practitioner for information; and the like. The initialize system step 505 may also include establishing communications with the TRUS signal processor 155, sending initialization commands to the TRUS signal processor 155, and retrieving configuration data values from it. The initialize system step 505 may further include retrieving configuration data values from memory within computer 165. Configuration data may include positioner 130 parameter data values; parameter data values related to the guide sheath 115, such as exit angle and position of the exit aperture 320 relative to the positioners' 130 “home” position; and parameters related to the needle, such as thickness and elasticity.

The device is positioned in step 510. In this step, the software 170 may send commands to the TRUS stepper 150, and any of the positioners 130, to position the device 105 in the cavity of the patient to acquire ultrasound imagery. Alternatively, if the TRUS stepper 150, or any of the positioners 130 are manually operated, the software 170 may prompt the practitioner to manually control the position of the device 105.

In step 515, the TRUS signal processor 155 processes ultrasound data acquired by the TRUS probe 110, and sends the processed ultrasound data to the computer 165 via data cable 160. The software 170 subsequently receives the processed ultrasound data, and displays the corresponding image. The software 170 may also store the processed ultrasound data values in memory. It will be apparent to one skilled in the art that step 515 may repeat continuously, whereby the practitioner may continuously be presented with real time ultrasound imagery throughout the surgical procedure.

After step 515, with the processed ultrasound image displayed, the practitioner may iteratively position the device and acquire imagery, substantially iterating steps 510 and 515 until the operator determines that the device 105 is properly oriented relative to the prostate, and that the support sheath 120 is properly positioned to stabilize the prostate during the subsequent steps of exemplary process 500.

In step 520, the practitioner may optimally position the TRUS probe 110 within the device 105 in order to obtain imagery of the prostate with sufficient image quality to enable positioning and insertion of the needle 125. In this step, the practitioner may enter user commands to the computer 165, which the software 170 converts into appropriate instructions that it sends to the TRUS stepper 150. One skilled in the art will readily recognize that the practitioner may enter user commands via a keyboard, mouse, trackball, or any other suitable computer input device. Alternatively, the practitioner may manually operate the TRUS stepper 150 while acquiring TRUS imagery by repeating step 515, until desired ultrasound imagery is attained. In accordance with the present invention, the support sheath 120 substantially stabilizes the cavity wall between the prostate and the TRUS probe 110, thereby mitigating variable forces on the prostate while the practitioner positions the TRUS probe 110 to acquire ultrasound imagery of appropriate quality for more accurate insertion of the needle 125.

In step 525, the practitioner enters user commands into the computer 170, which the software 170 converts into commands that the software 170 issues to either the guide sheath rotational positioner 145, the guide sheath translational positioner 140, or both. In response, the commanded positioners apply a force to the guide sheath 115 corresponding to the commands, and provide position and angle measurements corresponding to the new orientation of the guide sheath 115. When the relevant positioner completes the motion corresponding to the command, the software 170 retrieves position data or angle data from the relevant positioner or positioners, and stores the data values in a predetermined memory location.

In step 530, the software 170 executes instructions appropriate to implement a 3 DOF kinematic solution, which computes an estimated position of the needle 125 based on the angle and position data values respectively retrieved from the guide sheath rotational positioner 145; the guide sheath translational positioner 140; the needle depth positioner 135; and optionally from the TRUS stepper 150.

In step 530, the needle 125 may not have been inserted such that it protrudes through the corresponding exit aperture 320, since the guide sheath 115 is either moving, or has just been moved. As such, there may be no useful position data corresponding to the needle depth positioner 135. However, the practitioner may want to know what the projected path of the needle 125 will be, given the positions of the device 105 and the guide sheath 115. Accordingly, the software 170 may do the following: execute instructions to estimate a projected path of the needle 125; register this projected path to the processed ultrasound data; and display the projected path superimposed over the processed ultrasound imagery.

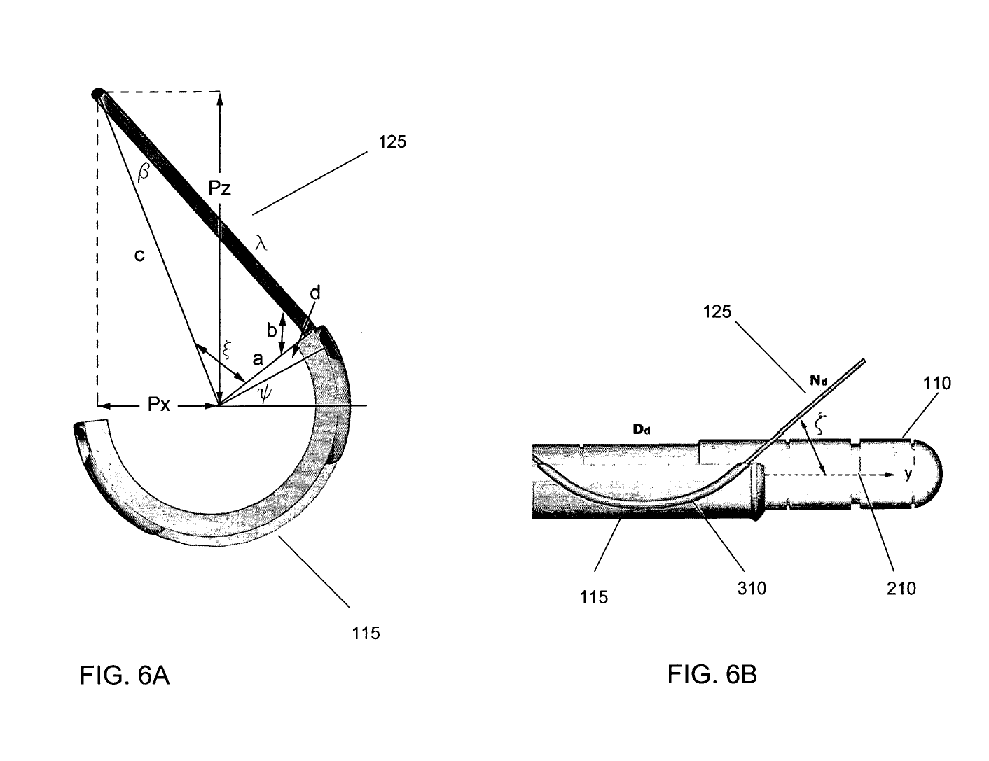

Generally, in estimating the position of the tip of the needle 125, the software 170 executes instructions to implement the following 3 DOF kinematic solution. As stated earlier, the degrees of freedom are: translation of the needle guide sheath in the cavity, D_{d}; rotation of the needle guide sheath inside the cavity, ψ, and insertion depth of the needle, N_{d}. FIG. 6A is a diagram showing a projection of the guide sheath 115 such that the longitudinal axis is extending into the page. FIG. 6B is a diagram showing a projection of the guide sheath from orthogonal to the longitudinal axis 210. The relationship between the needle exit angle and needle tip position is as follows:

$$ψ = arctan 2 ( {P}_{z} , {P}_{x} ) - ξ - d$$

$${N}_{d} = \frac{λ}{sin ( ζ )}$$

$${D}_{d} = {P}_{y} - {N}_{d} cos ( ζ )$$

where ζ is the exit angle, shown in FIG. 6B. It will be apparent to one skilled in the art that the equations above may be algebraically manipulated to allow one to compute the position Pₓ, P_{y}, P_{z}, given ψ, D_{d}, N_{d}, and implement the equations in computer instructions.

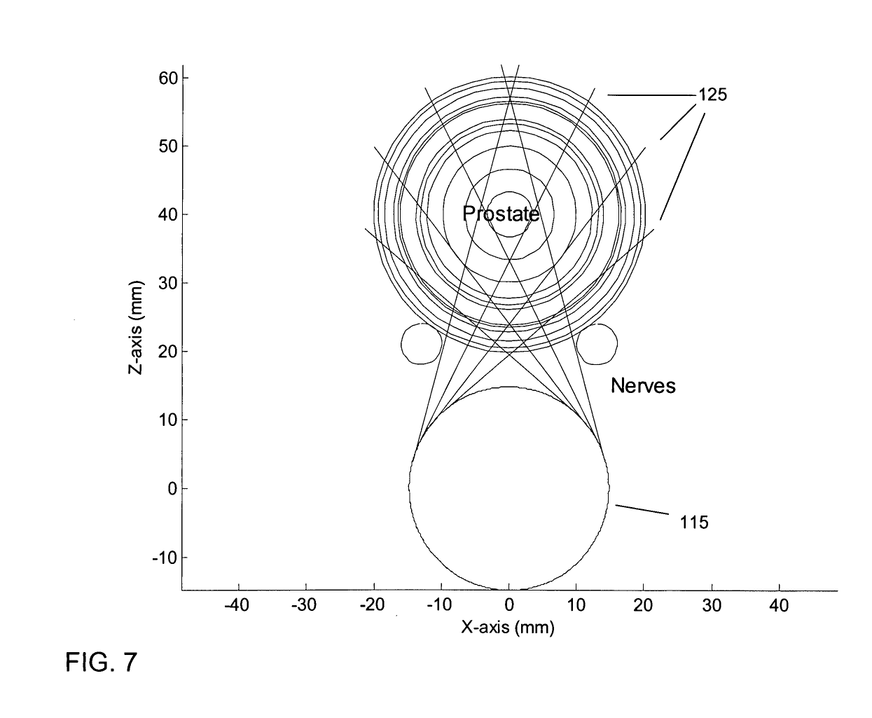

Repeating these equations for different values of N_{d} yields a set of needle tip positions as a function of needle depth. For each iteration, the software stores the computed needle tip position. The software 170 may then display each position, along with it's corresponding needle 125 depth, registered to the ultrasound imagery. This may provide the practitioner with an estimation of the needle tip position along with the corresponding needle depth required to establish that position, based on the current positions of the device 105 and the guide sheath 115. FIG. 7 illustrates an axial view of a prostate wherein various needle 125 positions are simultaneously projected given different guide sheath 115 positions.

According to step 535, if none of the projected needle tip positions are within a desired tolerance of the target within the prostate, the practitioner may repeat steps 525-535, thereby moving the guide sheath 115 to a new position, and again estimating a new set of projected needle tip positions as a function of needle depth.

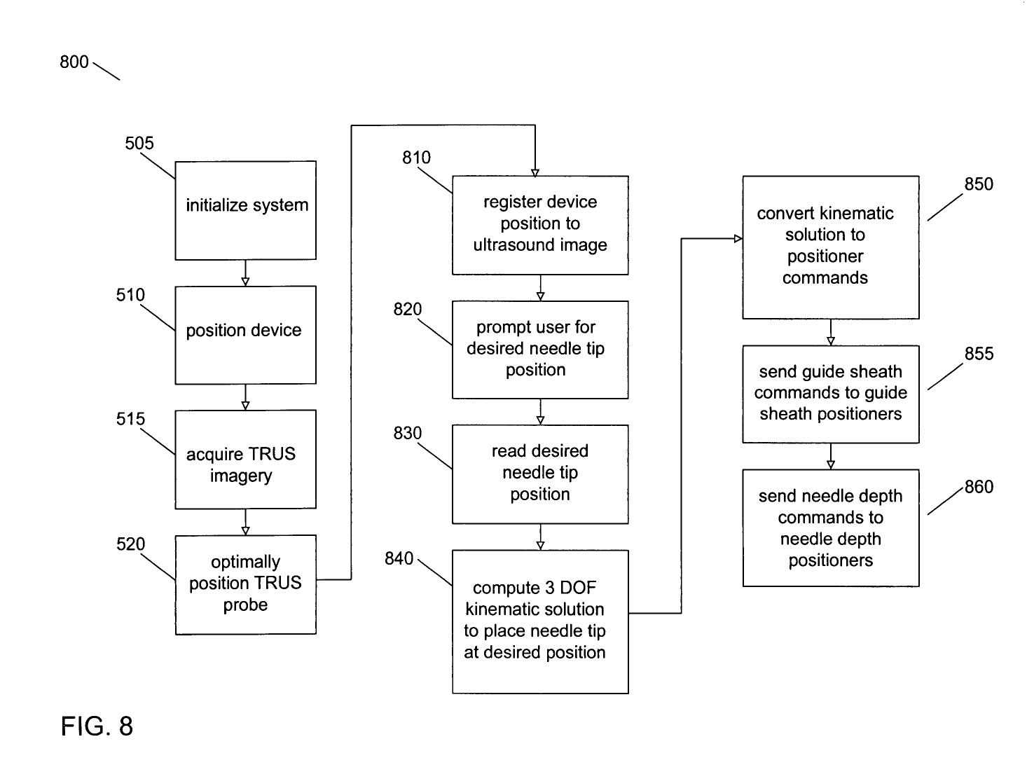

FIG. 8 illustrates an alternate exemplary process 800 for inserting a transcavital needle according to the present invention. As shown in process 800, steps 505-520 are substantially identical to the same-numbered steps in process 500, shown in FIG. 5. Accordingly, at the completion of step 520, the device 105 is positioned in the cavity, the TRUS probe 110 is positioned to provide desired ultrasound imagery of the prostate and the surrounding tissue, and the guide sheath 115 may be in its “home” position. In step 810, the software 170 retrieves the most recently stored processed ultrasound data values, and executes instructions to register the corresponding imagery to the most recently measured position of the TRUS probe 110, and optionally the most recently measured position of the guide sheath 115.

In step 820, the software 170 prompts the practitioner for a desired location for the tip of the needle 125. The practitioner may then select a desired needle tip position, based on the ultrasound image projected by the software 170. The software 170 may display a coordinate grid, or a cursor by which the practitioner may move the cursor to the desired needle tip position. The practitioner may input the desired location by clicking a mouse, or by entering the desired position coordinates with a keyboard. One skilled in the art will readily recognize that there are many ways by which a practitioner may be prompted for, and input, the desired needle tip position. In step 830, the software 170 reads the desired needle tip position that was input by the practitioner. The software 170 stores the desired position data values as corresponding to coordinates Pₓ, P_{z}, P_{z}, described above.

In step 840, the software 170 executes instructions to implement the equations above to compute a 3 DOF kinematic solution, taking the data values for Pₓ, P_{z}, P_{z}, coordinates and computing data values for the desired position of the guide sheath 115 in ψ, D_{d}, N_{d} coordinates. Then, in step 850, the software 170 executes instructions to convert the kinematic solution (ψ, D_{d}, N_{d}) to commands that will cause the positioners 130 to move the guide sheath and the needle to the desired needle tip position.

In step 855, the software 170 issues commands to the guide sheath rotational positioner 145 and the guide sheath translational positioner 140 according to the results of step 850. After the guide sheath positioners 140 and 145 establish the commanded positions, and report their respective positions to the software 170, the software 170 may proceed to step 860, in which the software issues commands to the needle depth positioner 135, computed in step 850, to place the needle tip at the desired position. If the needle depth positioner 135 operates manually, the software 170 may prompt the practitioner to insert the needle 125 to the depth computed in step 840. As the practitioner inserts the needle 125, the software 170 may iteratively send commands to the needle depth positioner 135, querying it for the latest depth measured by the positioner's encoder. The software 170 may then, using the depth measurements, iteratively compute the needle tip position using the above equations, register the position to the latest processed ultrasound data, and display the registered image with the computed needle tip position.

Exemplary process 500, or any combination of steps therein, and exemplary process 800, or any combination of steps therein, may be automated by including the appropriate computer instructions in software 170. For example, for steps 510-540, or for steps 510-860, software 170 may execute instructions for implementing a closed loop system. Such a closed loop system may include software modules for performing image processing for automated registration; comparison of projected needle tip position with desired position; estimation of needle tip position error; and updating and adjusting commands for positioners 130. It will be apparent to one skilled in the art that various control algorithms may implemented in software 170 for the purposes of automating and/or enhancing either process.

While the needle 125 is being inserted it is being subject to stresses as it bends according to the parametric curve of the needle guide 310. Depending on the elasticity of the needle 125, this curvature may be near or beyond the needle's elasticity properties. However, by rotating the needle 125 while it is being inserted, it is possible to bend the needle 125 beyond its elasticity without breaking it. Accordingly, the needle depth positioner 135 may include a rotational motor that rotates the needle 125 while the positioner inserts the needle 125. In this case, in step 540 or 860, the software 170 may command the rotational motor to rotate the needle 125 with a speed corresponding to the needle properties, which may be a configuration parameter valve stored in memory. Alternatively, if the needle depth positioner 135 is operated manually, the positioner may include a second handle, whereby the practitioner may rotate the needle 125 as it is inserted. Simultaneous rotation and insertion as described herein substantially distributes the plastic deformation energy nearly symmetrically along the helical path of the needle 125, which in turn substantially allows for a straight exit trajectory from the exit aperture 320.

It will be apparent to those skilled in the art that various modifications and variations of the exemplary embodiments described above can be made without departing from the spirit or scope of the present invention. Thus, it is intended that the present invention cover these modifications and variations provided they come within the scope of the appended claims and the equivalents thereof.

1. A transcavital needle insertion device comprising:
a support sheath;
an ultrasound probe; and
a guide sheath having at least one needle guide.

2. The device of claim 1, wherein the support sheath includes a plurality of holes.

3. The device of claim 1, wherein the ultrasound probe comprises a TRUS probe.

4. The device of claim 1, further comprising a stepper connected to the ultrasound probe.

5. The device of claim 1, wherein the guide sheath includes a partial cylindrical shape and wherein the guide sheath includes a longitudinal axis that is substantially collinear with an ultrasound probe longitudinal axis.

6. The device of claim 5, further comprising:
a first positioner means for controlling and measuring a first orientation of the guide sheath according to an angle around the longitudinal axis;
a second positioner means for controlling and measuring a second orientation of the guide sheath according to a distance along the longitudinal axis.

7. The device of claim 5, wherein the at least one needle guide includes an exit aperture having an exit axis central to the exit aperture, the exit axis having an exit angle relative to the longitudinal axis.

8. The device of claim 7, wherein the exit aperture includes a rounded shape.

9. The device of claim 7, wherein the exit aperture includes a cover.

10. The device of claim 7, further comprising a needle depth positioning means for controlling and measuring a length from a tip of a needle to the exit aperture.

11. The device of claim 7, wherein the needle depth positioning means includes a means for rotating the needle.

12. The device of claim 1, wherein the needle guide includes a curvature.

13. The device of claim 1, wherein the guide sheath comprises PTFE.

14. The device of claim 1, wherein the needle guide comprises stainless steel.

15. A method for inserting a needle into a cavity wall using a transcavital needle insertion device having a support sheath, an ultrasound probe, and a guide sheath having a needle guide, the method comprising the steps of:
inserting the transcavital needle device into a cavity;
obtaining an ultrasound image;
determining a target location inside the cavity wall;
computing a guide sheath position corresponding to the target location;
computing a needle depth corresponding to the target location and the guide sheath position;
positioning the guide sheath according to the guide sheath position; and
inserting the needle according to the needle depth.

16. The method of claim 15, wherein the step of inserting the transcavital needle device comprises the step of commanding a stepper to move the transcavital needle device a first distance into the cavity.

17. The method of claim 15, wherein the step of computing a guide sheath position comprises the steps of:
computing a translational position corresponding to a position along a longitudinal axis; and
computing an angular position corresponding to a rotation around the longitudinal axis.

18. The method of claim 17, wherein the step of positioning the guide sheath comprises the steps of:
sending a first command to a first positioner corresponding to the translational position; and
sending a second command to a second positioner corresponding to the angular position.

19. The method of claim 15, wherein the step of inserting the needle according to a needle depth includes the step of sending a needle depth command to the a needle depth positioner, the needle depth command corresponding to the needle depth.

20. The method of claim 19, wherein the step of inserting the needle further comprises sending a needle rotation command to the needle depth positioner.

21. The method of claim 20, wherein the needle rotation command corresponds to an elasticity of the needle.

22. A computer readable medium encoded with a program for controlling a transcavital needle insertion device, the device having a guide sheath, a guide sheath positioner, and a needle depth positioner, the program comprising the steps of:
acquiring a desired needle tip position;
converting the desired needle tip position to a desired translational position for the guide sheath, a desired rotational position for the guide sheath, and a desired needle depth;
sending a command to the guide sheath positioner corresponding to the desired translation position;
sending a command to the guide sheath positioner corresponding to the desired rotational position; and
sending a command to the needle depth positioner corresponding to the desired needle depth.

23. The computer readable medium of claim 22, wherein the step of acquiring a desired needle position comprises the step of:
prompting a practitioner for a desired needle position; and
reading a desired needle position data corresponding to the desired needle position, wherein the desired needle position data is input by the practitioner.

24. The computer readable medium of claim 23, wherein the step of acquiring a desired needle position further comprises the steps of:
acquiring an ultrasound image; and
displaying the ultrasound image, the displaying step containing an instruction to be executed before the step of prompting a practitioner.

25. The computer readable medium of step 22, wherein the step of sending a command to the needle depth positioner comprises the step of displaying information to a practitioner informing the practitioner to insert a needle to the desired needle depth.
